(19) United States
(12) Patent Application Publication
(10) Pub. No.: US 2018/0193231 A1
(43) Pub. Date: Jul. 12, 2018

(54) FAST DISINTEGRATING GRANULAR HAIR CLEANSING COMPOSITION

(71) Applicant: AMOREPACIFIC CORPORATION, Seoul (KR)

(72) Inventors: Ju A JUNG, Yongin-si, Gyeonggi-do (KR); Yang Gyu CHOI, Yongin-si, Gyeonggi-do (KR); Hyun Na KIM, Yongin-si, Gyeonggi-do (KR); Joon Ho BAE, Yongin-si, Gyeonggi-do (KR)

(73) Assignee: AMOREPACIFIC CORPORATION, Seoul (KR)

(21) Appl. No.: 15/740,339

(22) PCT Filed: Jun. 24, 2016
(86) PCT No.: PCT/KR2016/006756

§ 371 Date: Dec. 27, 2017

(30) Foreign Application Priority Data
Jun. 30, 2015 (KR) 10-2015-0092874
Jun. 23, 2016 (KR) 10-2016-0078679

(51) Int. Cl. A61K 8/02; A61K 8/19; A61K 8/362; A61K 8/73; A61K 8/34; A61Q 5/02

(52) U.S. Cl. CPC A61K 8/0275; A61K 8/19; A61K 8/362; A61K 2800/41; A61K 8/731; A61K 8/345; A61Q 5/02; A61K 8/732

(57) ABSTRACT

The present invention relates to a fast disintegrating granular hair cleansing composition in which a wet granulation process, which is a pharmaceutical preparation method, is introduced into a method for preparing a cleanser. The fast disintegrating granular hair cleansing composition is prepared by a wet granulation process so as to be readily disintegrated into granules by using small force, because of a low hardness thereof, and can easily form creamy foam since a large surface area comes into contact with water. In addition, portability, convenience of use and sanitation can be improved when molding is carried out in a single-dose in which the entire amount is used at one time.

### TECHNICAL FIELD

This application claims priority to and the benefit of Korean Patent Application No. 10-2015-0092874 filed on Jun. 30, 2015 and Korean Patent Application No. 10-2016-0078679 filed on Jun. 23, 2016, the disclosures of which are incorporated herein by reference in its entirety.

The present invention relates to a fast disintegrating granular hair cleansing composition which is rapidly disintegrated and has enhanced properties such as portability, sanitation and convenience of use.

### BACKGROUND ART

The contaminations of scalp and hair include secreted/excreted sebum and sweat, dead keratin cells, residues of hair cosmetics, and the like. When the contamination of the scalp becomes serious, hair follicles may be blocked, resulting in degraded function of hair papillae. In this case, the hair loss is caused because the normal growth of hair is interrupted. A shampoo is used to remove such materials so as to keep the hair clean, get rid of the materials with which skin pores are blocked so as to promote blood circulation, and supply oxygen to the hair and scalp so as to keep the hair and scalp healthy.

Various types of shampoos are commercially available on the market to respond to the consumers' needs. As one example, there are shampoos suitable for dry hair, greasy hair, dyed hair, neutral hair, damaged hair, and the like, depending on the types of hair. Also, there are functional shampoos having various functions, such as an anti-hairloss shampoo for preventing the hair loss, an anti-dandruff shampoo for removing dandruff, and the like.

Conventional shampoos are generally in the form of an ivory white liquid, and various types of simple-to-use shampoos such as a spray type, a solid type, and the like are also on the market.

A spray-type shampoo is referred to as a ‘dry shampoo,’ and is used by spraying it onto the scalp in the form of a spray. The spray-type shampoo is used to eliminate an overall sense of feeling greasy in the hair, and does not serve to perform actual cleaning of the hair and scalp.

A solid-type shampoo is prepared in the form of a shampoo bar, and has advantages in that it is simple to use and has nothing to leak like a liquid shampoo. However, the shampoo bar has problems in that it feels stiff after its use, collapses in shape as a use time increases because it is vulnerable to water, should be persistently rubbed with the hands to use it with water, and is not hygienic due to its repeated use.

In recent years, a foam-type solid shampoo referred to as a “bath balm” has been proposed in a new form of the solid-type shampoo.

A foam-type solid shampoo is prepared by mixing a foaming agent for generating foams, a surface detergent for washing, and a disintegrating agent with a binder to prepare a powder, and allowing the powder to lump through a process such as compression molding, and the like. The prepared foam-type solid shampoo is mixed with water before its use to generate bubbles, which are then used to clean the hair.

The foam-type solid shampoo has an advantage in that the problems regarding the collapse in shape caused in the conventional solid-type shampoos are solved, but has drawbacks in that a predetermined amount of time is taken until water used to generate foams penetrates into the solid shampoo, and the foams thus generated should be burst by applying a physical force to the foams.

### PRIOR ART DOCUMENT

### Patent Document

Korean Patent No. 10-123954 (Feb. 27, 2013), “Solid Shampoo Composition”

### DISCLOSURE

### Technical Problem

In order to solve the above problem, the present invention is designed by introducing a method for preparing pharmaceutical wet granules to prepare a cleaning agent. In this regard, the inventors of the present invention have found that a fast disintegrating granular hair cleansing composition may be readily disintegrated with a small force so that cleaning components are readily eluted when the composition is prepared by introducing a wet granulation process, which is the pharmaceutical dosage forming technique, to a process for preparing a cleaning agent, and also that portability, convenience of use, and sanitation may be improved when molding is carried out in a single unit dose. Therefore, the present invention has been completed based on these facts.

Accordingly, it is an object of the present invention to provide a fast disintegrating granular hair cleansing composition and a method for preparing the same.

### Technical Solution

According to an object of the present invention, there is provided a fast disintegrating granular hair cleansing composition, which includes a surfactant, a foaming agent, and a disintegrating agent, wherein granules are bound to each other by a binder to have a pill dosage form.

In addition, there is provided a method for preparing fast disintegrating granular hair cleansing composition, the method including:

(i) mixing a surfactant, a foaming agent, and a disintegrating agent to prepare a mixed powder;

(ii) adding a wetting agent to the mixed powder to perform kneading;

(iii) granule-forming and drying the resulting kneaded product to prepare granules;

(iv) mixing the granules with a binder; and

(v) compression-molding the resulting mixture using a mold.

### Advantageous Effects

The fast disintegrating granular hair cleansing composition according to the present invention can be readily decomposed when coming into contact with water, thereby forming creamy bubbles.

Also, the fast disintegrating granular hair cleansing composition, which is prepared by a wet granulation process and divided into a predetermined amount of aliquots which are then molded, can be readily disintegrated into granules with a small force, and a rapid disintegration process can be carried out due to a large surface area of the composition coming into contact with water.

In addition, portability, convenience of use and sanitation can be improved when the fast disintegrating granular hair cleansing composition of the present invention is molded in a single dose in which the entire amount of the composition is used at one time.

Further, when the fast disintegrating granular hair cleansing composition of the present invention is prepared by adding functional additives such as a hair conditioning agent, and the like, various additional functions such as stiff texture in use caused due to a powdery raw material can be improved or supplemented when in use.

### DESCRIPTION OF DRAWINGS

FIG. 1 is an image showing a shape of a fast disintegrating granular hair cleansing composition according to the present invention.

FIG. 2 is a flowchart illustrating the order of preparing the fast disintegrating granular hair cleansing composition according to the present invention.

FIG. 3 is an image showing a process for preparing the fast disintegrating granular hair cleansing composition according to the present invention.

FIG. 4 is data showing hardness of a fast disintegrating granular hair cleansing composition prepared in Example 1 of the present invention and hardness of a powdery cleaning agent prepared in Comparative Example 1 according to the drop distance.

FIG. 5 is an image showing shapes of the powdery cleaning agent of Comparative Example 1 of the present invention after the powdery cleaning agent is prepared, when the powdery cleaning agent is disintegrated, and when the powdery cleaning agent reacts with water.

FIG. 6 is an image showing shapes of the fast disintegrating granular hair cleansing composition of Example 1 of the present invention after the composition is prepared, when the composition is disintegrated, and when the composition reacts with water.

### BEST MODE

The present invention provides a fast disintegrating granular hair cleansing composition. The fast disintegrating granular hair cleansing composition is induced to be readily disintegrated with a small force due to low hardness thereof because a wet granulation process, which is a pharmaceutical preparation method, is introduced into a method for preparing a cleaning agent. Also, the fast disintegrating granular hair cleansing composition serves to reduce a disintegration time and enhance portability, sanitation, and convenience of use as well.

The term ‘fast-disintegrating foam type’ disclosed in the present invention refers to an easy conversion into bubbles in water, and the term ‘hair cleaning composition’ refers to a shampoo composition for cleaning hair.

Also, the ‘fast-disintegrating foam-type hair cleaning composition’ disclosed in the present invention is prepared into a pill dosage form. In this case, the composition may be referred to as a ‘pill-dosage-form solid shampoo,’ a ‘fast-disintegrating granular cleaning agent,’ a ‘fast-disintegrating granular cleaning agent,’ a ‘fast-disintegrating foam-type granular pill,’ a ‘foaming-pill solid shampoo,’ a ‘shampoo pill,’ or a ‘shampoo forming pill.’ Hereinafter, the composition may be referred to as a ‘fast-disintegrating granular cleaning agent’ for the sake of convenience.

The term ‘fast-disintegrating’ or ‘fast disintegration’ disclosed in this specification refers to a property of the fast-disintegrating granular cleaning agent provided in the present invention which is rapidly decomposed to generate foams. The fast-disintegrating property may be obtained by measuring a disintegration time taken until a fast-disintegrating granular cleaning agent having a diameter of 18 to 25 mm, preferably a diameter of 20 mm is completely decomposed at a temperature of 25 to 35° C., preferably a temperature of 30° C. In this case, when the disintegration time satisfies a value within 20 seconds, preferably 10 seconds, the fast-disintegrating granular cleaning agent exhibits a fast disintegrating property.

The term ‘hardness’ disclosed in this specification is associated with maintenance of a dosage form and texture in use thereof, and refers to a force required to disintegrate the fast-disintegrating granular cleaning agent. The hardness may be adjusted through the particle diameter and particle size distribution of a granular pill, and thus may be applied to set a force required for compression relative to the weight during molding. In the present invention, the hardness is measured using a texture analyzer (TA), and expressed as a value ‘g.’ In this case, the hardness has a value of 500 g or less, preferably 450 g or less and 50 g or more, and more preferably 300 g or less and 100 g or more. In this case, the higher value of the hardness refers to the higher force required to disintegrate the fast-disintegrating granular cleaning agent, and the lower value of the hardness means that the granular cleaning agent is disintegrated with a smaller force. However, when the hardness is too low, it is difficult to maintain the granular cleaning agent in the form of a pill. Therefore, it is desirable that the hardness satisfies the minimum value.

Hereinafter, the present invention will be described in further detail.

Fast Disintegrating Granular Hair Cleansing Composition

A fast-disintegrating granular cleaning agent is prepared by compressing the powder form obtained by mixing a surfactant for cleaning, a foaming agent for generating foams, and a disintegrating agent for disintegration with a binder, but has a drawback in that it is uncomfortable to use because water does not penetrate into the shampoo. Accordingly, in the present invention, the fast-disintegrating granular cleaning agent is prepared in the form of granules rather than the form of a powder, and the cleaning agent in the form of granules is compression-molded so that the cleaning agent is prepared in the form of a pill.

The fast-disintegrating granular cleaning agent, which is primarily prepared in the form of granules and allowed to lump, forms a number of sparse water channels to maintain at least a predetermined level of pores due to the presence of the granules in a structure thereof. Water rapidly penetrates and moves into the pores and the water channels made of the pores so that the collapse of the granules by water occurs over the entire pill dosage form at substantially the same time, thereby remarkably reducing a disintegration time. Also, the cleaning agent is readily disintegrated into granules even with a smaller force due to low hardness thereof, thereby further reducing the disintegration time.

First, a composition comprising the granules includes a surfactant, a foaming agent, and a disintegrating agent.

Specifically, the surfactant of the present invention is a composition that imparts a cleaning ability, and refers to a compound that includes a hydrophilic moiety easily soluble in water and a hydrophobic (lipophilic) moiety easily soluble in oil. The surfactant may be selected from an anionic surfactant, a cationic surfactant, an amphiprotic surfactant, a non-ionic surfactant, or a mixture thereof in consideration of the use of a cleaning agent. The anionic surfactant is dissociated into anions in water, the cationic surfactant is dissociated into cations, the amphiprotic surfactant is dissociated into anions or cations depending on the pH value of a solution because the amphiprotic surfactant has both anionic and cationic moieties in the molecule thereof, and the non-ionic surfactant may refer to a surfactant in which a hydrophilic moiety is a non-electrolytic, that is, unionized hydrophilic moiety, and may also include fatty acids in the form of an acid rather than the form of a salt.

Preferably, an alkyl sulfate having C12 (lauryl) to C18 (stearyl) carbon atoms, or a sulfate in which 2 to 5 polyoxyether groups are added to the alkyl sulfate may be used as the anionic surfactant having a good foaming ability and an excellent cleaning ability as well. In addition, an anionic surfactant having excellent foaming and cleaning abilities may be used in the composition of the present invention. Specifically, aliphatic sulfonates, aromatic sulfonates, alkyl sulfates, alkyl ether sulfates, alkyl sulfosuccinates, alkyl and acyl taurates, alkyl and acyl sarcosinates, sulfoacetates, C8 to C22 alkyl phosphates, alkyl phosphate esters and alkoxy alkyl phosphate esters, acyl lactates, C8 to C22 monoalkyl succinates and malates, sulfoacetates, acyl isethionates, polycarboxylates, and the like may be used as the anionic surfactant having excellent foaming and cleaning abilities.

For example, in addition to the anionic surfactant, an amphiprotic surfactant, a non-ionic surfactant, and the like may be mixed and used, and not particularly limited as long as the surfactants are used in a cleaning composition, and thus has an effect of improving a foaming ability. For example, alkyl betaines, alkyl sultaines, alkyl amido propyl betaines, alkyl amido propyl hydroxy sultaines, alkyl amphoacetates, alkyl diamphoacetates, and the like may be used as the amphiprotic surfactant. Also, the non-ionic surfactant that may be used herein may, for example, include alkyl glucose amides, alkyl glucose esters, polyoxyethylene esters, fatty acid alkylamides, alkyl amine oxides, alkyl polyglucosides, polyoxyethylene ester fatty acid, and the like.

To secure a cleaning effect, the surfactant may be preferably included at a content of 5 to 55% by weight, preferably 7 to 30% by weight, based on the total weight of the fast-disintegrating granular cleaning agent. When the content of the surfactant is less than this content range, a sufficient cleaning effect may not be secured. On the other hand, when the content of the surfactant is greater than this content range, the surfactant may be harmful to the skin due to an excessive cleaning ability thereof, and may increase probabilities of imparting soft and sticky texture in use and remaining in the skin or hair.

The foaming agent provided in the present invention serves to rapidly disintegrate a fast-disintegrating granular cleaning agent while forming bubbles on the principle in which an inorganic carbonate reacts with a weak acid to generate carbon dioxide (CO₂). Sodium bicarbonate (NaHCO₃), potassium bicarbonate (KHCO₃), calcium bicarbonate (Ca(HCO₃)₂), sodium carbonate (Na₂CO₃), potassium carbonate (K₂CO₃), calcium carbonate (CaCO₃), or a mixture thereof may be used as the inorganic carbonate. Preferably, sodium bicarbonate (NaHCO₃) may be used. Also, the weak acid may include one or more selected from citric acid, tartaric acid, phosphoric acid, acetic acid, maleic acid, fumaric acid, and the like, but the present invention is not limited thereto. Preferably, citric acid may be used.

The foaming agent may be included at a content of 10 to 50% by weight, preferably 15 to 45% by weight, based on the total weight of the fast-disintegrating granular cleaning agent. In this case, each of the inorganic carbonate and the weak acid may be used at a content of 5 to 45% by weight. More preferably, the inorganic carbonate and the weak acid may be desirably used at a weight ratio of 1:0.1 to 1:0.6 so as to generate carbon dioxide to secure a foaming ability. When the content of the foaming agent is less than this content range, it is difficult to sufficiently form bubbles, resulting in degraded texture in use. On the other hand, when the content of the foaming agent is greater than this content range, the texture in use may also be degraded due to excessive formation of bubbles.

The disintegrating agent provided in the present invention may be generally used in the pharmaceutical field, and a volume of the disintegrating agent expands in water. The disintegrating agent may include one or more selected from starches, microcrystalline cellulose, crospovidone, low-substituted hydroxypropyl cellulose, croscarmellose, and the like, but the present invention is not limited thereto.

The disintegrating agent may be used at a content of 1 to 25% by weight, preferably 5 to 20% by weight, based on the total weight of the fast-disintegrating granular cleaning agent. When the content of the disintegrating agent is less than this content range, a disintegration rate of a granular pill may be slow. On the other hand, when the content of the disintegrating agent is greater than this content range, it is not easy to adjust the disintegration rate.

The granules including the surfactant, the foaming agent, and the disintegrating agent may be prepared using a wet granulation process, which is a pharmaceutical preparation method as will be described below, and thus prepared in various shapes.

Preferably, the granules may be in a spherical or cylindrical shape (or a pellet shape). In this case, it is more desirable that the granules are in a cylindrical shape in consideration of water channel formation or pore characteristics. When the granules are in a spherical shape, the granules have a particle diameter of 0.1 to 3.0 mm, preferably 0.5 to 1.5 mm. Also, when the granules are in a cylindrical shape, the granules have a particle diameter of 0.1 to 3.0 mm, preferably 0.5 to 1.5 mm and a length of 0.2 to 5.0 mm, preferably 0.7 to 3.0 mm. When the diameter and length of the granules are less than these ranges, fluidity of the granules in a granulation process may be degraded, thereby causing lumping between the granules. On the other hand, the diameter and length of the granules are greater than these ranges, a large amount of time may be taken until the granules are completely dissolved in water due to a decrease in surface area of the granules coming into contact with water, and the creamy foaming ability may also be deteriorated. In this case, the size and shape of the granules may be adjusted in a granule forming process in which the granules are extruded through a sieve to prepare the granule during a wet granulation process.

The granules are bound by a binder to form a fast-disintegrating granular cleaning agent.

The binder is used to promote adhesion between the granules and maintain a state of the dosage form after compression. The binder that can be used herein may include glycerin, silicone oils, polyols, and a combination thereto. In this case, one or more selected from 1,3-butylene glycol, dipropylene glycol, propylene glycol, polyethylene glycol, and other liquid polyols may be used as the binder.

The content of the binder is limited so that predetermined levels of pores and water channels are maintained by a granular pill of the fast-disintegrating granular cleaning agent, wherein the pores and water channels are maintained in a final molding process. Preferably, the binder may be used at a content of 1 to 30% by weight, preferably 5 to 20% by weight, based on the total weight of the fast-disintegrating granular cleaning agent. When the content of the binder is less than this content range, binding or adhesion between the granules is difficult, resulting in an increased risk of granules being collapsed during a process of molding into pills. On the other hand, when the binder is used at an excessive amount greater than this content range, the cleaning agent may not secure a fast disintegration property due to a decrease in the pores and water channels, and may have a soft and sticky texture in use.

Also, the fast-disintegrating granular cleaning agent of the present invention may further include additives such as a thickening agent for adjusting the viscosity of the aforementioned cleaning agent composition or a bubble stabilizing agent for stabilizing bubbles, when necessary. The thickening agent is dissolved in water or organic solvent to form a high-viscosity sticky substance, and is used to enhance the viscosity of a cleaning agent. For example, bentonite, clay, silica, an acrylic polymer, a vinyl-based polymer, cellulose, and gum may be used as the thickening agent, and lauric acid may be used as the bubble stabilizing agent.

When the fast-disintegrating granular cleaning agent of the present invention is a hair cleaning agent, the fast-disintegrating granular cleaning agent may further include a hair conditioning agent for imparting luster, softness, antistaticity and elasticity to the hair. The hair conditioning agent may include glycerin, guarquat as a cationic polymer, C10 to C22 alkyl trimethyl ammonium chloride (ATAC), C10 to C22 dialkylmethylammonium chloride (DDAC), behenyltrimethylammonium chloride (BTAC), stearyltrimethylammonium chloride (STAC), tricetylmethylammonium chloride (TCMAC), and the like, which may be used alone or in combination thereof. In the case of conventional liquid hair cleaning agents, the textures of raw materials themselves do not need to be contemplated because a composition is dissolved. However, because the fast-disintegrating granular cleaning agent of the present invention in which a powdery raw material is granulated into solids has a stiff texture in use characteristic of the powder, the hair conditioning agent may be additional added thereto to improve the texture in use.

Also, in the fast-disintegrating granular cleaning agent of the present invention, one or more fillers selected from lactose, mannitol, starch, sorbitol, crystalline cellulose, polypyrrolidone, hydroxymethyl cellulose, and the like may be used as a component which does not deteriorate innate cleaning and foaming abilities of the cleaning agent so as to impart a proper sense of mass and achieve filling and molding in a mold.

In addition, the fast-disintegrating granular cleaning agent of the present invention may include additives generally used in the related art, for example, one or more additives selected from a preservative, a fragrance, a pH control agent, a viscosity adjusting agent, a moisturizing agent, and a disinfectant. The additives may be, for example, included at a content of 0.001 to 10% by weight, based on the total weight of the fast-disintegrating granular cleaning agent of the present invention. In this case, the contents of the additives may vary depending on the types of the respective additives.

When the fast-disintegrating granular cleaning agent of the present invention having the composition as described above comes into contact with water, fast disintegration occurs as water rapidly penetrates into inner/outer parts of the granular pills. In this case, the granular pills may be readily disintegrated into granules even with a small force due to low hardness thereof, and creamy bubbles may be then generated. The fast-disintegrating granular cleaning agent is dividedly molded in a single dose, preferably molded at a single dose of 1 to 5 g, more preferably a single dose of 2 to 3 g. In this case, after the fast-disintegrating granular cleaning agent comes into contact with water having a temperature of 25 to 35° C., a small force is applied thereto, and small granules rapidly react with water to form bubbles. In this case, the fast-disintegrating granular cleaning agent has a disintegration time of 5 to 15 seconds, preferably 10 seconds or less.

The fast-disintegrating granular cleaning agent may be prepared so that the single dose is less than or greater than this dose range in consideration of the length or density of the hair. As one example, when the fast-disintegrating granular cleaning agent is prepared into a spherical pill dosage form, the fast-disintegrating granular cleaning agent may be prepared with a diameter of 18 to 25 pi (Φ). For short hair, 2 to 3 g of the fast-disintegrating granular cleaning agent may be used to prepare a spherical pill dosage form having a diameter of 20 to 22 pi. At this time, one pi (Φ) refers to a diameter of 1 mm, and the single-dose fast-disintegrating granular cleaning agent means that the fast-disintegrating granular cleaning agent may have a diameter of 18 to 25 mm.

Also, the use and shape of the fast-disintegrating granular cleaning agent according to the present invention are not limited. For example, the fast-disintegrating granular cleaning agent may have any shapes as long as it can be prepared using a pharmaceutical wet granulation method. As one example, the fast-disintegrating granular cleaning agent may be prepared in a spherical shape, an oblate shape, a semi-spherical shape, a cylindrical shape, a rugby ball shape, a conical shape, a barrel shape, a polygonal shape, a trapezoidal shape, a prismatic shape, a cubic shape, and the like. In this case, any shapes of the fast-disintegrating granular cleaning agent are not limited as long as such shapes can impart a feeling of satisfaction and an excellent texture in use to users. As one example, the fast-disintegrating granular cleaning agent is prepared in a spherical shape according to one exemplary embodiment of the present invention.

Additionally, to stabilize the dosage form against moisture, the fast-disintegrating granular cleaning agent may also be coated with oil having water resistance, or material such as a lubricant or silica to form a thin film on the dosage form. Also, a packing material is preferably selected to stabilize articles against moisture and maintain the hardness in the packages. For example, a multi-pocket-type packing material in which a number of dosage forms are packaged, a dispenser-packing material in which one dosage form is discharged with one touch, or a peel-off-type plastic packing material (e.g., a blister package) may be applied.

Method for Preparing Fast-Disintegrating Granular Cleaning Agent Hair Cleaning Composition

The fast-disintegrating granular cleaning agent according to the present invention has a structure in which granules are bound by a binder, and is particularly prepared through a pharmaceutical wet granulation method.

A wet granulation process is performed in the order of process of weighing and mixing raw materials, a combination process, an assembly process, a drying process, and a finishing process. The wet granulation process has an advantage in that fluidity of the granules may be secured, a density of the raw materials increases, and compressibility of the granular cleaning agent may be improved. In the present invention, the aforementioned surfactant, foaming agent, disintegrating agent and other additives, all of which are used to clean hair, are first used as the raw materials in the wet granulation process to prepare granules, and a binder is used to assemble the granules in order to prepare a single-dose fast-disintegrating granular cleaning agent.

Particularly, the process for preparing the fast-disintegrating granular cleaning agent of the present invention is mainly divided into a granulation step and a molding step. Because disintegration occurs when the fast-disintegrating granular cleaning agent comes into contact with water, these processes are performed to prepare an anhydrous dosage form in which the use of water is excluded.

Specifically, the fast-disintegrating granular cleaning agent is prepared through the following steps:

(i) mixing a surfactant, a foaming agent, and a disintegrating agent to prepare a mixed powder;

(ii) adding a wetting agent to the mixed powder to perform kneading;

(iii) granule-forming and drying the resulting kneaded product to prepare granules;

(iv) mixing the granules with a binder; and

(v) compression-molding the resulting mixture using a mold.

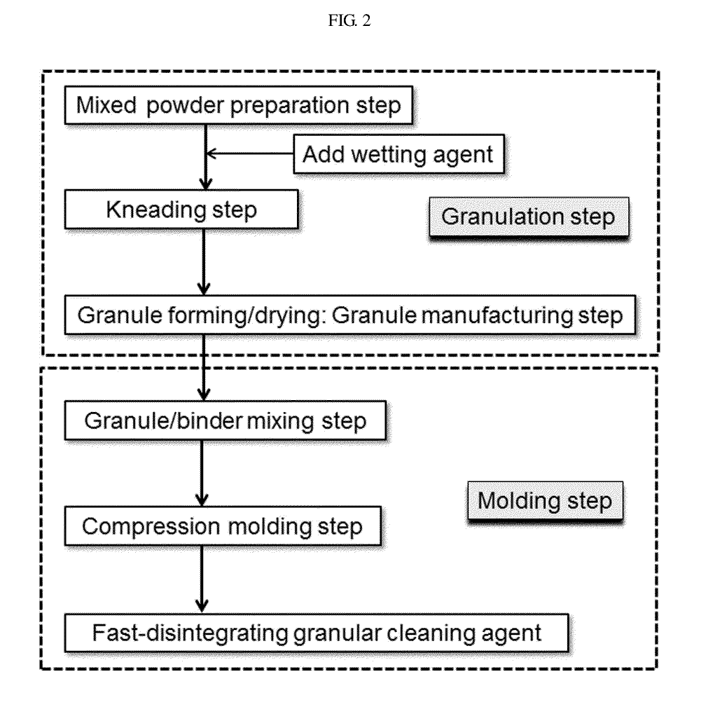

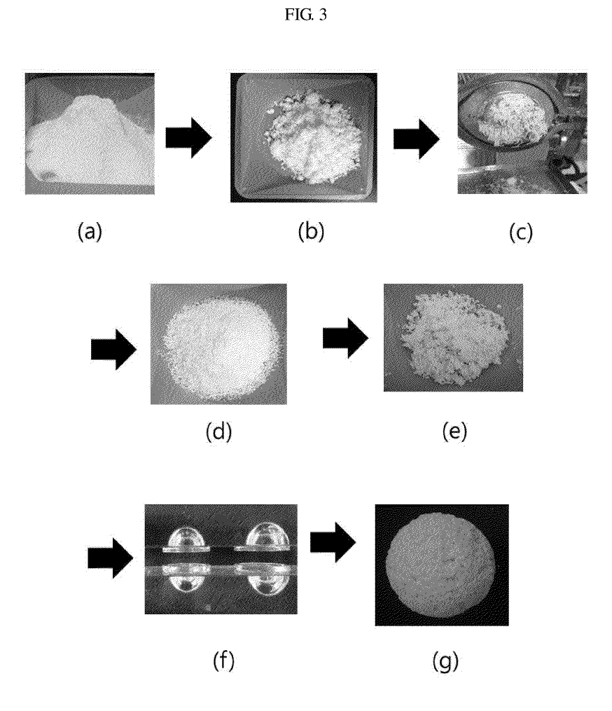

Hereinafter, the respective steps of the preparation method will be described in detail with reference to the accompanying drawings. FIGS. 2 and 3 are a flowchart and an image showing the order of preparing the fast disintegrating granular hair cleansing composition according to the present invention, respectively.

(i) Mixed Powder Preparation Step

First, a surfactant, a foaming agent, and a disintegrating agent are added to a mixer, and uniformly mixed to prepare a mixture (see FIG. 3A).

In this case, the respective components are used in the same content ranges as in the aforementioned components.

(ii) Kneading Step

Next, a wetting agent is added to the mixture obtained in step (i), and subjected to a kneading process to prepare a paste (see FIG. 3B).

The wetting agent provided herein is used to promote physical binding between the surfactant, the foaming agent and the disintegrating agent. In particular, when the surfactant, the foaming agent and the disintegrating agent come into contact with water, a reaction (disintegration) occurs immediately. In the present invention, an anhydrous binder solution excluding water is used as a liquid substance for uniformly mixing the raw materials and binding the raw materials. One or more solvents selected from ethanol, methanol and acetone, all of which have relatively low toxicity and are volatile, may be used as the anhydrous binder solution. Preferably, ethanol may be used.

The wetting agent may be used at a content of 1 to 50 parts by weight, preferably 5 to 25 parts by weight, based on a total of 100 parts by weight of the sum of the granular components. When the content of the wetting agent is less than this content range, the shape of the granule may collapse due to the insufficient binding between the raw materials when the granules are prepared. On the other hand, when the wetting agent is used at an excessive amount greater than this content range, it is impossible to prepare the granules.

The kneading process may be performed using a known kneading machine. As one example, a kneader or pony mixer may be used.

(iii) Granule Preparation Step

Subsequently, after the paste kneaded in step (ii) is passed through a screen, the paste is subjected to an extrusion process, a granule forming process, and a drying process to prepare granules (See FIGS. 3C and D).

At this time, a sieve having a mesh size of 0.5 to 1.5 is generally used as the screen, and the particle diameter of the granules finally prepared may vary depending on the mesh size of the sieve.

The drying may be performed at a temperature at which the wetting agent in the granules may be sufficiently removed, preferably performed at 25 to 80° C. for 0.5 to 5 hours. To remove the wetting agent, the temperature and time may be varied by a person having ordinary skill in the art.

A granulator generally used in the pharmaceutical field may be used as a granulator for preparing the granules of the present invention, but the present invention is not particularly limited thereto.

(iv) Granule/Binder Mixing Step

Then, a binder is added to the granules obtained in step (iii), and uniformly mixed (see FIG. 3E).

The binder may be used by weighing the raw material as described aboive. Also, the binder may be added at once or several times in divided doses.

At this time, a mixer is not particularly limited in the present invention. For example, a high-speed mixer, a double cone mixer, a cone blender, a ribbon blender, a shovel mixer, a pug mixer-Henschel mixer, a Brabender mixer, a twin-screw kneader, and the like may be used as the mixer.

(v) Compression Molding Step

Then, the mixture obtained in step (iv) is compression-molded using a mold to prepare a fast-disintegrating granular cleaning agent in a pill dosage form (see FIGS. 3F and G).

The compression molding may be performed by injecting the mixture into a mold and applying a pressure to the mixture. At this time, the pressure may be applied to an extent in which the mixture is sufficiently bound to be molded into a predetermined shape. The pressure may vary depending on the size of the mold. When the applied pressure is not sufficient, the fast-disintegrating granular cleaning agent may be damaged or broken when used or stored. Therefore, the fast-disintegrating granular cleaning agent has to be handled carefully.

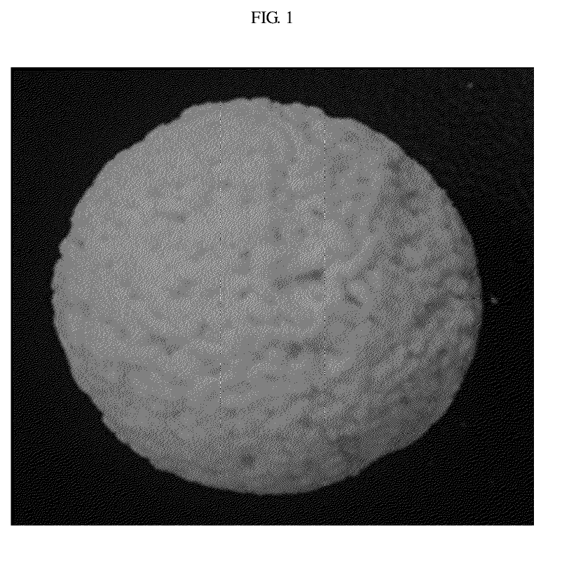

The mold may be used as long as the fast-disintegrating granular cleaning agent can be molded into a pill dosage form, as shown in FIG. 1.

The fast-disintegrating granular cleaning agent prepared through the aforementioned steps is prepared using a modified pharmaceutical wet granulation process. Therefore, the fast-disintegrating granular cleaning agent is in the form in which the granules comes into lump rather than the form in which powders are bound to each other as known in the art. As a result, random pores are formed on the surface and inside of the fast-disintegrating granular cleaning agent, and water channels are formed so that the cleaning agent is readily broken into small granules with a small force when the granules come into contact with water. Then, these small granules are rapidly disintegrated to form creamy bubbles.

Therefore, the fast-disintegrating granular cleaning agent has excellent properties such as spread ability on hands, texture, and the like because a user may simply use the fast-disintegrating granular cleaning agent to form bubbles.

### Mode for Invention

Hereinafter, the present invention will be described in further detail with reference to embodiments thereof. However, it will be apparent to those skilled in the art that the following examples given herein are not intended to limit the scope of the present invention, and various changes and modifications can be made to the examples of the present invention without departing from the scope of the present invention.

### EXAMPLES

### Example 1

A fast-disintegrating granular pill was prepared using the composition listed in the following Table 1. In this case, the respective processes are shown in the image of FIG. 3.

1. Granulation Step

Sodium lauryl sulfate (SLS), citric acid, sodium bicarbonate, starch, a fragrance, STAC 80(I), lactose, and guarquat were uniformly mixed, and ethanol was added thereto. Thereafter, the mixture in the form of a paste was extruded into a predetermined size through a sieve with a mesh size of 1.0 mm, and then dried to prepare dry granules from which ethanol was removed.

2. Molding Step

Glycerin and silicone oil were added to the prepared dry granules, and mixed. Thereafter, the resulting mixture was extruded in a spherical mold to prepare a molded fast-disintegrating granular cleaning agent (diameter: 20 pi).

### Comparative Example 1

Sodium lauryl sulfate (SLS), citric acid, sodium bicarbonate, starch, a fragrance, STAC 80(I), lactose, and Guarquat were uniformly mixed, and then glycerin and silicone oil were added thereto. Thereafter, the resulting mixture was extruded in a spherical mold to prepare a molded powdery cleaning agent.

TABLE 1

| | | Comparative |
|---|---|---|
| Components (% by weight) | Example 1 | Example 1 |
| Sodium lauryl sulfate (SLS) | 13.4 | 15 |
| Citric acid | 6.35 | 7 |
| Sodium bicarbonate | 22.7 | 25 |
| Starch | 6.97 | 7.5 |
| Fragrance | 0.13 | 0.15 |
| STAC 80(I) | 0.45 | 0.5 |
| lactose | 31 | 33.85 |
| Guarquat | 2.3 | 2.5 |
| Glycerin | 6.8 | 7.5 |
| Silicone oil | 0.9 | 1 |
| Ethanol | 9.0 | — |

### Experimental Example 1

The average disintegration hardness, underwater disintegrating shapes, and underwater disintegration times of the fast-disintegrating granular cleaning agent (Example 1) compression-molded into the granules and the powdery cleaning agent (Comparative Example 1) compression-molded into the powders were tested. The results are listed in the following Table 2.

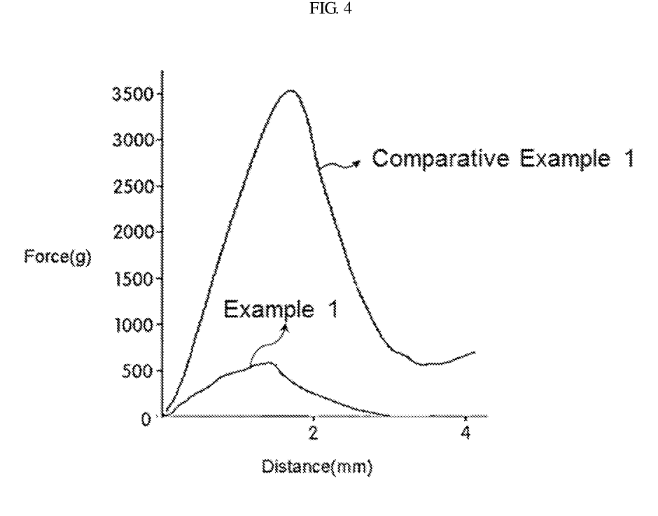

The average disintegration hardness was measured using a texture analyzer. Here, a test speed was set to 2.00 mm/sec, a target mode was set to a distance of 5.0 mm, and a trigger type was set to an Auto Force of 0.049 N. A force required to disintegrate a pill was converted into a mass (g), and a force converted according to the drop distance was indicated on the data shown in FIG. 4 In this case, the lower value of the hardness measured means that the cleaning agent is readily collapsed with a smaller force. Also, the disintegrating shape and the disintegration time were measured at a temperature of 30° C. (in water).

TABLE 2

| Test items | Example 1 | Comparative Example 1 |
|---|---|---|
| Average disintegration | 375 | 3,250 |
| hardness (g) | | |
| Disintegrated shape | Readily disintegrated | Lumped into |
| | into granules | undissolved mass |
| Disintegration time | 10 seconds | 20 minutes |

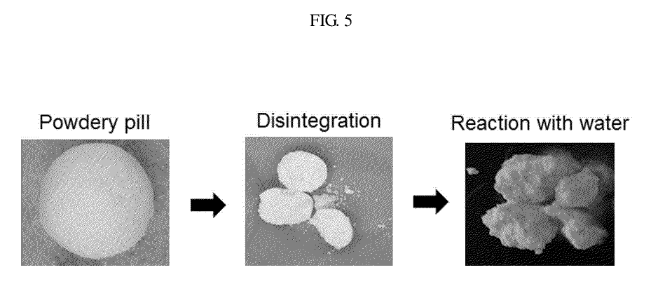

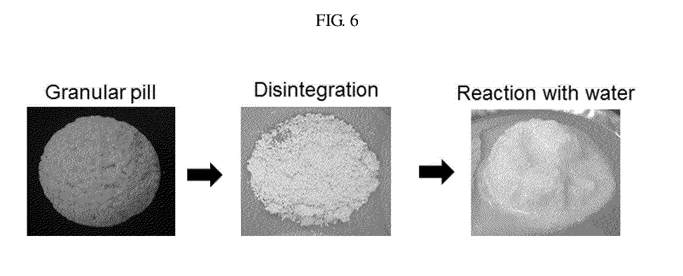

As listed in Table 2 and shown in FIG. 4, it can be seen that the fast-disintegrating granular cleaning agent (Example 1) was readily disintegrated with a small force because the average disintegration hardness of the fast-disintegrating granular cleaning agent was 375 g which was approximately 1/10 lower than the disintegration hardness (3,250 g) of the powdery cleaning agent (Comparative Example 1). In this case, the images of the disintegrated cleaning agents were shown in FIGS. 5 and 6.

Also, the underwater disintegrated shapes are shown in FIGS. 5 and 6. Here, it was confirmed that the granular cleaning agent (Example 1) was very readily disintegrated, compared to the powdery cleaning agent (Comparative Example 1), and the disintegration time of the granular cleaning agent (Example 1) was 10 seconds, the value of which was 1/120 shorter than the disintegration time (20 minutes (=1,200 seconds)) of the powdery cleaning agent (Comparative Example 1).

FIG. 5 is an image showing shapes of the powdery cleaning agent of Comparative Example 1 of the present invention after the powdery cleaning agent is prepared, when the powdery cleaning agent is disintegrated, and when the powdery cleaning agent reacts with water. After the powdery cleaning agent of Comparative Example 1 was prepared, the shape of the powdery cleaning agent had a dense and compact surface. At this time, a force expressed as a mess of 3,200 g was required until the powdery cleaning agent was disintegrated, and the powdery cleaning agent was disintegrated into large masses. As result, it was confirmed that, because water did not easily penetrated into the disintegrated large masses, a large amount of time was taken until the large masses was completely dissolved in water, and the powdery cleaning agent exhibited sticky texture in use like porridge.

Meanwhile, FIG. 6 is an image showing shapes of the fast disintegrating granular hair cleansing composition of Example 1 of the present invention after the composition is prepared, when the composition is disintegrated, and when the composition reacts with water. After the fast-disintegrating granular cleaning agent of Example 1 was prepared, pores were formed on a surface of the fast-disintegrating granular cleaning agent and formed in the fast-disintegrating granular cleaning agent. At this time, it was confirmed that a force expressed as a mess of 300 g was required until the fast-disintegrating granular cleaning agent was disintegrated, the small granules rapidly reacted with water to form bubbles in water, and the fast-disintegrating granular cleaning agent had a very excellent effect in spreading on hands and forming bubbles.

Because the fast-disintegrating granular cleaning agent of the present invention is molded into solids while maintaining pores between the granules due to the granulation in a preparation process, the fast-disintegrating granular cleaning agent has significantly low hardness, compared to the conventional powder-compressed solid cleaning agents. Also, because lumping between particles hardly occurs and the particles exhibits excellent fluidity when the fast-disintegrating granular cleaning agent is disintegrated or dissolved in water, the disintegration time can be shortened. Therefore, the fast-disintegrating granular cleaning agent of the present invention can be useful in improving portability, convenience of use, and sanitation when the fast-disintegrating granular cleaning agent is dividedly molded in a single dose.

1. A fast disintegrating granular hair cleansing composition comprising a surfactant, a foaming agent, and a disintegrating agent, wherein granules are bound to each other by a binder to have a pill dosage form.

2. The fast disintegrating granular hair cleansing composition of claim 1, wherein the fast disintegrating granular hair cleansing composition comprises 5 to 55% by weight of the surfactant, 10 to 50% by weight of the foaming agent, 1 to 25% by weight of the disintegrating agent, and 1 to 30% by weight of the binder, based on the total weight of the composition.

3. The fast disintegrating granular hair cleansing composition of claim 1, wherein the surfactant comprises one selected from the group consisting of an anionic surfactant, a cationic surfactant, an amphiprotic surfactant, a non-ionic surfactant, and a mixture thereof.

4. The fast disintegrating granular hair cleansing composition of claim 1, wherein the foaming agent comprises an inorganic carbonate and a weak acid.

5. The fast disintegrating granular hair cleansing composition of claim 4, wherein the inorganic carbonate and the weak acid are included at a weight ratio of 1:0.1 to 1:0.6.

6. The fast disintegrating granular hair cleansing composition of claim 4, wherein the inorganic carbonate comprises one selected from the group consisting of sodium bicarbonate (NaHCO₃), potassium bicarbonate (KHCO₃), calcium bicarbonate (Ca(HCO₃)₂), sodium carbonate (Na₂CO₃), potassium carbonate (K₂CO₃), calcium carbonate (CaCO₃), and a mixture thereof.

7. The fast disintegrating granular hair cleansing composition of claim 4, wherein the weak acid comprises one selected from the group consisting of citric acid, tartaric acid, phosphoric acid, acetic acid, maleic acid, fumaric acid, and a mixture thereof.

8. The fast disintegrating granular hair cleansing composition of claim 1, wherein the disintegrating agent comprises one selected from the group consisting of starches, microcrystalline cellulose, crospovidone, low-substituted hydroxypropyl cellulose, croscarmellose, and a mixture thereof.

9. The fast disintegrating granular hair cleansing composition of claim 1, wherein the binder comprises one selected from the group consisting of glycerin, silicone oils, polyols, and a mixture thereof.

10. The fast disintegrating granular hair cleansing composition of claim 1, wherein the granules have a particle diameter of 0.5 to 1.5 mm.

11. The fast disintegrating granular hair cleansing composition of claim 1, wherein the pill dosage form has a particle diameter of 18 to 25 mm.
